(19) United States
(12) Patent Application Publication
(10) Pub. No.: US 2005/0148526 A1
(43) Pub. Date: Jul. 7, 2005

(54) Methods of obtaining isoform specific expression in mammalian cells

(76) Inventors: Malgorzata Anna Kisielow, Bellinzona (CH); Sandra Kleiner, Weissenborn (DE); Yoshikuni Nagamine, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/502,235

(22) PCT Filed: Jan. 22, 2003
(86) PCT No.: PCT/EP03/00611

(30) Foreign Application Priority Data
Jan. 23, 2002 (GB) 0201477.7

(51) Int. Cl. A61K 48/00; C12Q 1/68; C12N 15/85

(52) U.S. Cl. 514/44; 435/6; 435/455; 435/325

(57) ABSTRACT

A method is provided for isoform specific gene expression in a mammalian cell in the absence of other isoforms. The method uses RNAi to achieve the specific expression.

### FIELD OF INVENTION

The present invention relates to the field of gene expression, in particular to inhibiting the expression of a specific isoform of a gene in mammalian cells using double-stranded RNA and to expressing a specific isoform in a clean back ground. The double-stranded RNA based technology of the invention has wide applications, such as for determining function of a particular gene isoform or developing therapeutic methods for treating diseases.

### BACKGROUND OF THE INVENTION

Many eukaryotic genes are expressed as multiple isoforms through the differential utilization of transcription and translation initiation sites or through alternative splicing, thus giving rise to proteins of related sequence but with biochemically as well as biologically distinct features (Andreadis et al., 1987, Annu. Rev. Cell Biol. 3, 207-242). Although, in many cases, multiple isoforms are expressed in the same cell at the same time, the expression level and pattern of each isoform may vary with cell type and developmental stage, making the study of each isoform confusing and difficult.

The conventional approach for investigating the function of individual isoforms requires conditions where only one isoform is expressed or eliminated to provide a clean background (i.e. without the influence of other isoforms). Thus, typically the desired isoform has been expressed in cells or whole organisms in which the target gene has been deleted, a process that is time consuming. While several protocols for tissue-specific expression or elimination of a gene of interest have been developed (Johnson et al., 1989, Mol. Cell Biol. 9, 3393-3399; Tan, 1991, Dev. Biol. 146, 24-37; Gorman and Bullock, 2000, Curr. Opin. Biotechnol. 11, 455-460), reports of isoform-specific gene inactivation (Migliaccio et al., 1999, Nature 402, 309-313) or expression in a clean background are limited. Such protocols are usually lengthy, often requiring several months to establish the desired conditions. During this time, cells or organisms may adapt to the new conditions (Muller, U., 1999, Mech. Dev. 82, 3-21) and caution is required when interpreting the results.

In the last few years, advances in nucleic acid chemistry and gene transfer have inspired new approaches to engineer specific inhibition of gene expression. Antisense technology has been the most commonly described approach in protocols to achieve gene-specific inhibition, although more recently targeted gene silencing based on double-stranded RNA (dsRNA) inducing a response called post-transcriptional gene silencing (PTGS) or RNA interference (RNAI) has been proposed. PTGS is a phenomenon originally reported in plants (van der Krol et al., 1990, Plant Cell 2, 291-299; Napoli, et al., 1990, Plant Cell 2, 931-943), where introduction of a transgene causes silencing of the endogenous homologous gene as well as itself. The mechanism of PTGS involves enhanced mRNA degradation with double-stranded (ds)RNA as the trigger (Cogoni and Macino, 2000, Curr. Opin. Genet. Dev. 10, 638-643; Carthew, 2001, Curr. Opin. Cell Biol. 13, 244-248). A similar phenomenon (quelling) has been observed in Neurospora (Cogoni et al., 1994, Antonie Van Leeuwenhoek 65, 205-209). The mechanism underlying RNAi has been partially elucidated and a 21- to 23-nt-long dsRNA was found to be the intermediate/mediator of mRNA decay (Zamore et al., 2000, Cell 101, 25-33; Bernstein et al., 2001, Nature 409, 363-366).

In the animal kingdom, dsRNA-mediated gene silencing was first described in the nematode C. elegans (Fire et al., 1998, Nature 391, 806-811) and was termed RNA interference (RNAi). Subsequently, RNAi has been observed in a wide range of organisms including flies, trypanosomes, hydra, zebrafish and mice oocytes (Cogoni and Macino, 2000; Bosher and Labouesse, 2000, Nat. Cell Biol. 2, E31-36). Although RNAi has been used extensively in non-mammalian systems, dsRNA inhibition has only recently been applied successfully in mammalian systems. For example, an efficient posttranscriptional gene-silencing method has been reported that employs a small interfering double-stranded RNA (siRNA) in mammalian cells (Elbashir et al., 2001, Nature 411, 494-498). The authors describe that transfection of 21-nucleotide dsRNA (siRNA) can trigger PTGS of both the co-transfected and the endogenous gene in cultured mammalian cells.

Using a cell-free system of dsRNA-mediated mRNA decay, mRNA was shown to be cleaved in Drosophila embryonal cell extracts within the region of identity with the dsRNA (Zamore et al., 2000). However, the potency of RNAi in worms and flies suggests that dsRNA acts catalytically and/or is amplified (Zamore, 2001, Nature Structural Biology 8, 746-750). Although it has been suggested that siRNAs are not replicated by endogenous RNA-dependent RNA polymerase in C. elegans, data now exists supporting a model whereby siRNAs indeed act as primers for cellular polymerases and are extended to form ds RNAs in both C. elegans and Drosophila (Lipardi et al., 2001, Cell 107, 297-307; Sijen et al., 2001, Cell 107, 465-476; Nishikura, 2001, Cell 107, 415-418). Propagation of RNAi to other regions of the mRNA in this way would result in the spread of gene silencing to related sequences, thereby not allowing isoform-specific dsRNA inhibition.

There is a need in the art for a system to evaluate gene function, in particular for the evaluation of isoform function in mammalian cells, as well as for therapies dependent on dsRNA inhibition of closely related mRNAs in mammalian cells and this invention meets that need.

Relevant Literature

1 Andreadis, A., Gallego, M. E. and Nadal-Ginard, B. (1987) Generation of protein isoform diversity by alternative splicing: mechanistic and biological implications. Annu. Rev. Cell Biol. 3, 207-242

2 Johnson, J. E., Wold, B. J. and Hauschka, S. D. (1989) Muscle creatine kinase sequence elements regulating skeletal and cardiac muscle expression in transgenic mice. Mol. Cell Biol. 9, 3393-3399

3 Tan, S. S. (1991) Liver-specific and position-effect expression of a retinol-binding protein-lacZ fusion gene (RBP-lacZ) in transgenic mice. Dev. Biol. 146, 24-37

4 Gorman, C. and Bullock, C. (2000) Site-specific gene targeting for gene expression in eukaryotes. Curr. Opin. Biotechnol. 11, 455-460

5 Migliaccio, E., Giorgio, M., Mele, S., Pelicci, G., Reboldi, P., Pandolfi, P. P., Lanfrancone, L. and Pelicci, P. G. (1999) The p66shc adaptor protein controls oxidative stress response and life span in mammals. Nature (London) 402, 309-313

6 Muller, U. (1999) Ten years of gene targeting: targeted mouse mutants, from vector design to phenotype analysis. Mech. Dev. 82, 3-21

7 Luzi, L., Confalonieri, S., Di Fiore, P. P. and Pelicci, P. G. (2000) Evolution of Shc functions from nematode to human. Curr. Opin. Genet. Dev. 10, 668-674

8 Nakamura, T., Muraoka, S., Sanokawa, R. and Mori, N. (1998) N-Shc and Sck, two neuronally expressed Shc adapter homologs. Their differential regional expression in the brain and roles in neurotrophin and Src signaling. J. Biol. Chem. 273, 6960-6967

9 Bonfini, L., Migliaccio, E., Pelicci, G., Lanfrancone, L. and Pelicci, P. G. (1996) Not all Shc's roads lead to Ras. Trends Biochem. Sci. 21, 257-261

10 Migliaccio, E., Mele, S., Salcini, A. E., Pelicci, G., Lai, K. M., Superti-Furga, G., Pawson, T., Di Fiore, P. P., Lanfrancone, L. and Pelicci, P. G. (1997) Opposite effects of the p52shc/p46shc and p66shc splicing isoforms on the EGF receptor-MAP kinase-fos signalling pathway. EMBO J. 16,706-716

11 van der Krol, A. R., Mur, L. A., Beld, M., Mol, J. N. and Stuitje, A. R. (1990) Flavonoid genes in petunia: addition of a limited number of gene copies may lead to a suppression of gene expression. Plant Cell 2, 291-299

12 Napoli, C., Lemieux, C. and Jorgensen, R. A. (1990) Introduction of a chimeric chalcone synthase gene in petunia results in reversible cosuppression of homologous genes in trans. Plant Cell 2, 931-943

13 Cogoni, C. and Macino, G. (2000) Post-transcriptional gene silencing across kingdoms. Curr. Opin. Genet. Dev. 10, 638-643

14 Carthew, R. W. (2001) Gene silencing by double-stranded RNA. Curr. Opin. Cell Biol. 13, 244-248

15 Cogoni, C., Romano, N. and Macino, G. (1994) Suppression of gene expression by homologous transgenes. Antonie Van Leeuwenhoek 65, 205-209

16 Fire, A., Xu, S., Montgomery, M. K., Kostas, S. A., Driver, S. E. and Mello, C. C. (1998) Potent and specific genetic interference by double-stranded RNA in Caenorhabditis elegans. Nature (London) 391, 806-811

17 Bosher, J. M. and Labouesse, M. (2000) RNA interference: genetic wand and genetic watchdog. Nat. Cell Biol. 2, E31-36

18 Zamore, P. D., Tuschl, T., Sharp, P. A. and Bartel, D. P. (2000) RNAi: double-stranded RNA directs the ATP-dependent cleavage of mRNA at 21 to 23 nucleotide intervals. Cell 101, 25-33

19 Bernstein, E., Caudy, A. A., Hammond, S. M. and Hannon, G. J. (2001) Role for a bidentate ribonuclease in the initiation step of RNA interference. Nature (London) 409, 363-366

20 El shir, S. M., Harborth, J., Lendeckel, W., Yalcin, A., Weber, K. and Tuschl, T. (2001) Duplexes of 21-nucleotide RNAs mediate RNA interference in cultured mammalian cells. Nature (London) 411, 494-498ba

21 Lipardi, C., Wei, Q. and Paterson, B. M. (2001) RNAi as random degradative PCR: siRNA primers convert mRNA into dsRNAs that are degraded to generate new siRNAs. Cell 107, 297-307

22 Sijen, T., Fleenor, J., Simmer, F., Thijssen, K. L., Parrish, S., Timmons, L., Plasterk, R. H. and Fire, A. (2001) On the Role of RNA Amplification in dsRNA-Triggered Gene Silencing. Cell 107, 465-476

23 Cohen, S. S. (1968) Virus-induced Enzymes, Columbia University Press, New York

24 Fagard, M. and Vaucheret, H. (2000) Systemic silencing signal(s). Plant Mol. Biol. 43, 285-293

25 Tuschl, T., Zamore, P. D., Lehmann, R., Bartel, D. P. and Sharp, P. A. (1999) Targeted mRNA degradation by double-stranded RNA in vitro. Genes. Dev. 13, 3191-3197

### SUMMARY OF THE INVENTION

The present invention provides a method of expressing a desired isoform of a gene product in a cell absent undesired isoforms of the gene product, comprising the steps of:

(a) exposing a mammalian cell to at least one nucleic acid that comprises at least a partially double-stranded ribonucleic acid, the double-stranded portion having at least 95% sequence identity, preferably 100% sequence identity, to a common nucleic acid sequence shared by two or more isoforms of the gene product; and

(b) introducing an expression vector encoding a desired isoform of the gene product into the mammalian cell, the nucleic acid encoding the desired isoform having a sequence comprising one or more mismatches relative to the double-stranded portion of the nucleic acid, operably linked to a promoter capable of driving expression of the desired isoform in the cell. Preferably, the common nucleic acid sequence is at least 19 consecutive nucleotides in length and is common to all endogenous isoforms of the gene product in the cell of interest.

The nucleic acid is preferably 19 to 25 nucleotides long and the double-stranded portion of the nucleic acid is preferably 100% identical to the common nucleic acid sequence. In particular, the at least partially double-stranded ribonucleic acid preferably contains a double-stranded portion of at least 19 nucleotides, more preferably of 19 nucleotides, and at least one single-stranded 3′ overhang of two-nucleotides, preferably both upper and lower strands having a single-stranded 3′ overhang of two-nucleotides.

The desired isoform is encoded by a sequence comprising at least one mismatch relative to the double-stranded portion of the nucleic acid to ensure that the nucleic acid of at least partially double stranded RNA character does not affect expression of the desired isoform. The desired isoform can therefore be encoded by a sequence having one, two or more mismatches relative to the double-stranded portion of the nucleic acid as long as the altered coding sequence still encodes the desired product. Thus, at least one codon (e.g., two or more) encoding the desired isoform may differ from the endogenously used codon at the same position. It is well known in the art that many amino acids are designated by more than one triplet codon. Preferably, the desired isoform has an identical protein sequence to the corresponding endogenous isoform (unless an endogenous wild-type sequence is used to correct an endogenous mutant isoform, in which case the wild-type sequence is used), although amino acid substitutions, deletions or additions to the isoform sequence may be tolerated, in particular conservative substitutions.

In one embodiment, the desired isoform replaces a mutant isoform expressed by the cell, allowing correct gene function. The mutant isoform can be oncogenic, apoptotic, tumor suppressive, inflammation inducive or suppressive, or angiogenic, for example.

In a further embodiment, the method of the invention is used to determine the function of the desired isoform (i.e, isoform of unknown function).

The method is useful in all types of mammalian cells, including cancer cells, such as HeLa (cervical cancer), PC3 (prostate cancer), MDA-MB-231 (breast cancer) and MCF-7 (breast cancer), or cells in vivo.

The desired isoform can be transcribed under the control of any promoter, such as an endogenous promoter, a constitutive promoter, an inducible promoter or a tissue-specific promoter.

The invention also provides methods of assigning function to a desired isoform of unknown (or unrecognized) function. Also provided are kits and materials for carrying out the invention, as well as cells exhibiting isoform-specific expression in a clean genetic background. The invention also encompasses therapeutic uses and compositions based on the methods of the invention.

### DETAILED DESCRIPTION OF THE INVENTION

In its broadest aspect, the present invention provides a method of expressing a desired isoform of a gene product in a cell absent undesired isoforms of a gene product, by exposing a mammalian cell to at least one nucleic acid, the nucleic acid being at least a partially double-stranded ribonucleic acid and the double-stranded portion having at least 95% sequence identity to a common nucleic acid sequence shared by two or more isoforms of the gene product; and introducing an expression vector encoding a desired isoform of the gene product into the mammalian cell, the desired isoform having a sequence comprising one or more mismatches relative to the double-stranded portion of the nucleic acid, operably linked to a promoter capable of driving expression of the desired isoform in said cell.

The nucleic acid will typically be a ribonucleic acid (RNA) that in double-stranded form has at least 95%, preferably 98% most preferably 100% sequence identity to a common nucleic acid sequence shared by two or more isoforms of the gene product. Preferred RNA molecules for inhibition comprise sequences identical to a common nucleic acid sequence shared by two or more isoforms of the gene product over at least 15-25 consecutive bases, preferably over at least 19 consecutive bases. Preferably the common nucleic acid sequence is common to all endogenous isoforms of the gene product expressed by the cell of interest. The RNA sequence is preferably chosen to have identity with exon sequences. Gene expression is inhibited in a sequence-specific manner in that nucleotide sequences corresponding to the duplex region of the RNA are targeted for inhibition.

Sequence identity (and therefore specificity) may be optimized and common nucleic acid sequence determined by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Sequence specificity is important to ensure that the nucleic acid does not cross-react and affect expression of other unrelated gene sequences.

The nucleic acid will typically be relatively short for use in mammalian cells to allow efficient delivery of the nucleic acid to the cell and also to avoid any protein kinase R (PKR) response of the cell. Alternatively, other ways of circumventing non-specific cellular responses to dsRNA or more efficient delivery methods can be used. Thus, the nucleic acid is at least partially double-stranded RNA in character and will typically be 18 to 25 nucleotides long. The double-stranded RNA character is provided by sequences at least 18 nucleotides long, preferably 19 nucleotides in length, which are preferably 100% identical to the common nucleic acid sequence shared by two or more isoforms of the desired gene. In preferred embodiments, the nucleic acid comprises at least one single-stranded 3′ overhang of two-nucleotides, preferably both upper and lower strands having a single-stranded 3′ overhang of two-nucleotides.

The at least partially double-stranded RNA can be formed by a self-complementary RNA strand (such as a transcript having an inverted repeat), or by two or more complementary RNA strands. RNA duplex formation may be initiated either inside or outside the cell. The RNA is introduced in an amount that allows delivery of at least one copy per cell, preferably at least 5, 10, 100, 500 or 1000 copies per cell, depending on the application. The amount introduced is dependent on the desired effect and can be easily determined empirically.

The nucleic acid may comprise nucleotides or linkages other than those that occur naturally in ribonucleic acid, for example, to stabilize the dsRNA from degradation, especially when RNA is delivered to a cell and not produced by the cell. Thus, one can employ oligoribonucleotides or oligonucleotides that comprise one or more modified (i.e., synthetic or non-naturally occurring) nucleotides. Usually, nucleotide monomers in a nucleic acid are linked by phosphodiester bonds or analogues thereof. Analogues of phosphodiester linkages include phosphorothioate, phosphorodithioate, phosphoroselenoate, phosphorodiselenoate, phosphoroanilothioate, phosphoranilidate, phosphoramidate, peptide, and the like linkages. Those of skill in the art will recognize that the reagents employed are commercially available or, in the case of the oligonucleotides, can be prepared using commercially available instrumentation. Preferably the duplex RNA will comprise ribonucleotide units or other nucleotide units that allow appropriate processing by the cell and efficient inhibition of the isoforms expressed by the cell. The nucleic acid (typically RNA) may be synthesized either in vivo or in vitro, particularly in vitro when short sequences are employed. Endogenous RNA polymerase of the cell may mediate transcription in vivo, or cloned RNA polymerase can be used for transcription in vivo or in vitro, essentially as described below.

Thus, in one step of the method of the invention a mammalian cell is exposed to the nucleic acid described above to inhibit expression of specific isoforms expressed in a cell of interest, preferably to inhibit all related isoforms. The term “isoform” is well known in the art and is meant to encompass gene products that are produced as a result of differential gene splicing as well as from the use of alternative transcription and translation start sites. In addition, for the purpose of the present invention, the term isoforms include any closely related sequences and therefore may include a mutated gene in a cell, such as deleterious point mutations and the like. The mutant isoform can be oncogenic, apoptotic, tumor suppressive, inflammation inducive or suppressive, or angiogenic, for example, and its deleterious function corrected by the method of the invention. Although the Examples below illustrate the invention using ShcA, any gene product produced in multiple forms can be targeted using the methods of the present invention. For example, such proteins may be therapeutically important proteins, such as enzymes, e.g., kinases (PKB), ras, integrins, E2F, Rb, FGF, other signaling molecules and transcription factors.

The effect of dsRNA on gene expression will typically result in expression of the target isoforms being inhibited by at least 10%, 33%, 50%, 90%, 95% or 99% or more when compared to a cell not treated by this step.

The mammalian cell can be any cell of interest. Thus, the cell may be cells from the inner cell mass, extraembryonic ectoderm or embryonic stem cells, totipotent or pluripotent, dividing or non-dividing, parenchyma or epithelium, immortalized or transformed, or the like. The cell may be a stem cell or a differentiated cell. Cell types that are differentiated include without limitation adipocytes, fibroblasts, myocytes, cardiomyocytes, endothelium, dendritic cells, neurons, glia, mast cells, blood cells and leukocytes (e.g., erythrocytes, megakaryotes, lymphocytes, such as B, T and natural killer cells, macrophages, neutrophils, eosinophils, basophils, platelets, granulocytes), epithelial cells, keratinocytes, chondrocytes, osteoblasts, osteoclasts, hepatocytes, and cells of the endocrine or exocrine glands, as well as sensory cells. Preferably for the study of biological function of a particular isoform or for studying protein-protein interactions, cells that are easily cultured are preferred. These may include cancer cells, for example, including HeLa (cervical cancer), PC3 (prostate cancer), MDA-MB-231 (breast cancer) and MCF-7 (breast cancer) cells. It will be apparent to one of ordinary skill in the art that the teachings of the specification can easily be applied to other situations, such as cancer cells for the replacement of a mutated “isoform” with one or more desired isoform(s).

In a further step, an expression vector encoding a desired isoform of the gene product is introduced into the mammalian cell, the desired isoform having a sequence comprising one or more mismatches relative to the double-stranded portion of the nucleic acid, operably linked to a promoter capable of driving expression of the desired isoform in said cell. The nucleic acid encoding the desired isoform preferably comprises a sequence comprising one or more mismatches, preferably two or more, relative to the double-stranded portion of the nucleic acid used to inhibit expression of the endogenous isoforms to ensure that the nucleic acid (RNAi) does not inhibit expression of the desired isoform. Preferably, the expression vector encodes a desired isoform using at least one codon, more preferably at least two codons, that differ(s) from the endogenous sequence coding the corresponding isoform. Changes in protein sequence between the endogenous isoform and desired isoform are also encompassed by the present invention, in particular those substitutions, deletion or additions that do not affect gene function, which are preferably conservative substitutions. Most preferably, the desired isoform has an identical protein sequence to the corresponding endogenous isoform (although the coding sequence will be different due to codon usage).

For transcription of a transgene in vivo (whether the desired isoform or si/dsRNA, optionally in the form of a hairpin structure), an expression construct comprising at least one regulatory region (e.g., promoter, enhancer, silencer, splice donor and acceptor and polyadenylation signal) operably linked to the DNA coding for the desired RNA transcript(s) may be used to transcribe the RNA strand (or strands). The promoter can be of almost any origin. Preferred are promoters that are active in the chosen host cells like the SV40, beta-actin, metallothionein, T7, polyhedrin and cytomegalovirus promoters. The promoter can be a constitutive promoter, an inducible promoter or a tissue-specific promoter, for example, allowing inhibition to be targeted to an organ or cell type; or transcription to be induced upon stimulation of an environmental condition (e.g., infection, stress, temperature, chemical inducers); and/or engineering transcription at a developmental stage or age. Alternatively, a knock-in construct can be used to transcribe the nucleic acid of interest under the control of an endogenous promoter, as is known in the art. Modified or unmodified RNA can also be chemically or enzymatically synthesized by manual or automated reactions as described above. The RNA may be synthesized by a cellular RNA polymerase or a bacteriophage RNA polymerase (e.g., T3, T7, SP6).

Expression vectors may also include sequences allowing for their autonomous replication within the host organism, sequences that encode genetic traits that allow cells containing the vectors to be selected, and sequences that increase the efficiency with which the RNA is transcribed. Stable long-term vectors may be maintained as freely replicating entities by using regulatory elements of viruses. Cell lines may also be produced which have integrated the vector into the genomic DNA and in this manner the transcript(s) is/are produced on a continuous basis.

Thus, an expression vector can further include, if desired, additional sequences operably linked to a promoter, such as a reporter gene (e.g., fluorescent proteins, e.g., green fluorescent protein, β-galactosidase, alkaline phosphatase, luciferase, CAT, selective gene markers that facilitate the selection of transformants due to the phenotypic expression of the marker gene (e.g., those expressing antibiotic resistance or, in the case of auxotrophic host mutants, genes which complement host lesions), or other useful sequences.

Nucleic acids (whether expression vectors or double-stranded RNA) can be introduced into a cell by various standard methods in genetic engineering, including physical methods, for example, simple diffusion, by injection of a solution containing the nucleic acid, bombardement by particles covered by the nucleic acid, soaking the cell or organism in a solution of the nucleic acid, or electroporation of cell membranes in the presence of the nucleic acid. A particularly preferred method for delivering nucleic acids is the use of electroporation. Alternatively, a viral construct accomplishes both efficient introduction of an expression construct into a cell and transcription of RNA encoded by the expression construct. Other methods known in the art for introducing nucleic acids to cells may also be used, as is apparent to the artisan, such as lipid-mediated delivery systems, chemical mediated transport, such as calcium phosphate transfection, DEAE-dextran transfection, and the like. The transfected host cells can be cultured by standard methods in cell culture.

The methods of the present invention are particularly useful for determining the function of a particular isoform. In this aspect of the invention, the method further comprises identifying a phenotype of the mammalian cell compared to when the desired isoform is absent, and assigning the phenotype or function to the desired isoform.

The invention also provides a kit comprising reagents useful in the methods of the invention, which may include a nucleic acid being at least a partially double-stranded ribonucleic acid and the double-stranded portion having at least 95% sequence identity to a common nucleic acid sequence shared by two or more isoforms of the gene product; and an expression vector encoding a desired isoform of the gene product, the desired isoform having a sequence comprising one or more mismatches relative to the double-stranded portion of the nucleic acid, operably linked to a promoter capable of driving expression of the desired isoform in said cell. The preferred particulars of the components used in the method are those described hereinabove.

A mammalian cell exhibiting isoform-specific expression achieved by any of the methods of the invention is also provided.

As the isoform may be used to correct aberrant isoforms, also provided is a method for treating a disease comprising administering to a subject in need of treatment an effective amount of a nucleic acid being at least a partially double-stranded ribonucleic acid and the double-stranded portion having at least 95% sequence identity to a common nucleic acid sequence shared by two or more isoforms (in particular the aberrant isoform) of the gene product; and an expression vector encoding a desired isoform of the gene product, the desired isoform having a sequence comprising one or more mismatches relative to the double-stranded portion of the nucleic acid, operably linked to a promoter capable of driving expression of the desired isoform in said cell. Therefore. The invention provides the use of the nucleic acid as hereinabove described for the treatment of aberrant isoform expression and for the manufacture of a medicament.

Administration of pharmaceutical compositions of the invention may be accomplished orally or parenterally. Methods of parenteral delivery include topical, intra-arterial (e.g. directly to the tumour), intramuscular, subcutaneous, intramedullary, intrathecal, intraventricular, intravenous, intraperitoneal, or intranasal administration. In addition to the active ingredients, these pharmaceutical compositions can contain suitable pharmaceutically acceptable carriers comprising excipients and other compounds that facilitate processing of the active compounds into preparations which can be used pharmaceutically. Further details on techniques for formulation and administration can be found in the latest edition of Remington's Pharmaceutical Sciences (Maack Publishing Co, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, etc, suitable for ingestion by the patient.

Pharmaceutical preparations for oral use can be obtained through combination of active compounds with solid excipient, optionally grinding a resulting mixture, and processing the mixture of granules, after adding suitable additional compounds, if desired, to obtain tablets or dragee cores. Suitable excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores can be provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterise the quantity of active compound (i.e. dosage).

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds can be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The invention is further described, for the purposes of illustration only, in the following examples. Methods of molecular genetics, protein and peptide biochemistry and immunology referred to but not explicitly described in this disclosure and examples are reported in the scientific literature and are well known to those skilled in the art.

### EXAMPLES

### Example 1

### Inhibition (Knock-Down) of ShcA in HeLa Cells

The signaling adaptor/scaffold protein ShcA is a member of the Shc family, which consists of three genes, ShcA, ShcB/Sli/Sck and ShcC/N-Shc/Rai (Luzi et al., 2000, Curr. Opin. Genet. Dev. 10, 668-674). ShcA is ubiquitously expressed, whereas ShcB and ShcC are expressed specifically in the brain (Nakamura et al., 1998, J. Biol. Chem. 273, 6960-6967). There are three isoforms of ShcA, p66ShcA, p52ShcA and p46ShcA, derived from a single gene through differential usage of transcription initiation sites (p66 versus p52/p46) and translation start sites (p52 versus p46), which differ only in the amino terminal regions (Luzi et al., 2000). We chose ShcA to exemplify the invention as different cellular functions have been suggested for each isoform and therefore is of scientific interest to obtain isoform-specific expression of ShcA. However, it will be apparent to one of ordinary skill in the art that the invention is not limited in any way to this particular gene family.

The primary transcript of p52/p46 mRNA contains the entire sequence of p66 mRNA; however, the very 5′ region of p66 mRNA is present in the first intron of p52/p46 mRNA but is absent in the latter mRNA, having been spliced out. The p46 and p52 isoforms are derived from the same mRNA using different translation initiation sites. The p66-siRNA site is in the 5′ region only present in p66 ShcA mRNA, while h/m-shc siRNA site is in the second exon which is present in both mRNAs. Note that the sequence of p66-shc siRNA is from a 5′ region of human p66ShcA mRNA and is absent in p52/46 ShcA mRNA.

Cells were transfected using the OligofectAMINE Reagent without siRNA or with h/m-shc siRNA and luc siRNA. Briefly, HeLa cells were cultured in Dulbecco's Modified Eagle's Medium (DMEM, Gibco-BRL) supplemented with 10% (v/v) fetal calf serum (AMIMED), 0.2 mg/ml streptomycin and 50 units/ml penicillin at 37° C. in a humidified CO₂ (5%) incubator. One day before transfection with siRNA, cells were plated in 6-well plates in media without antibiotics at 1.4×10⁵ cells per well.

The following 21-mer oligoribonucleotides pairs were used as siRNA: h/m-shc siRNA from nt 677-697 (in the PTB domain), 5′-CUA CUU GGU UCG GUA CAU GGG-3′ (SEQ ID NO:1) and 5′-CAU GUA CCG AAC CAA GUA GGA-3′ (SEQ ID NO:2). Sequences were derived from the sequence of human p66ShcA mRNA (accession number: HSU7377) and its complement and each pair has a 3′ overhang of 2 nt on each side. Designed RNA oligonucleotides were blasted against Database (GEMBL) to ensure gene specificity. The RNA oligonucleotides were obtained from Microsynth (Balgach, Switzerland). Annealing was performed according to Elbashir et al. (2001, Nature 411, 494-498). The complementary two strands (each at 20 μM) in 200 μl of annealing buffer (100 mM potassium acetate, 30 mM HEPES-KOH pH 7.4,2 mM magnesium acetate) were heated for 1 min at 90° C. and then incubated for 1 h at 37° C. An siRNA corresponding to nucleotides 753-773 of the firefly luciferase mRNA (luc siRNA) was used as a negative control.

siRNAs were introduced into HeLa cells using the OligofectAMINE Reagent (Life Technologies) according to the manufacturer's instructions, with 10 μl of 20 μM siRNA and 3 μl of transfection reagent per well. At different times after transfection, whole-cell extracts were prepared and analyzed by Western blotting. Cells were lysed in a buffer containing 120 mM NaCl, 50 mM Tris pH 8.0, and 1% NP40 plus Complete (Roche) protein inhibitor tablets. The whole-cell extracts (20 μg) were analyzed by Western blotting for Shc A, β-tubulin and Grb2 using a polyclonal rabbit anti-Shc antibody (1:250; Transduction Laboratories), mouse monoclonal anti-Grb2 (1:1000; Transduction Laboratories) or a mouse monoclonal anti-β tubulin antibody (1:1000; Sigma). We used anti-rabbit or anti-mouse HRP-linked antibodies from Amersham as secondary antibodies. An enhanced chemiluminescence detection method (ECL; Amersham) was employed and the membrane was exposed to Kodak Xomat LS film. Quantification of ShcA proteins was done using ImageQuant 5.0.

When HeLa cells were transfected with the h/m-shc siRNA, the levels of all three ShcA isoforms were strongly reduced 24 h after transfection and reached less than 20% of the control after 48 h and 4% after 60 h. The level of control protein β-tubulin was not affected under the conditions employed, and this was also the case for Grb2, a protein that specifically interacts with ShcA upon activation of growth-factor signaling (Luzi et al., 2000). Luc siRNA had no effect. Time-course analysis showed that the levels of all ShcA isoforms remained low until the fifth day after transfection but started to increase thereafter. The reduction in the three isoforms was uniform, suggesting that both p52/p46 and p66 ShcA mRNAs were equally targeted by the siRNA. According to the invention, repeated transfection maintains or re-establishes the low levels of ShcA isoforms.

### Example 2

### Isoform-Specific Knock-Down

The selective inhibition of the ShcA p66 isoform is demonstrated in this example using siRNA specific for p66. Cells were left untreated (control) or transfected without siRNA (control) or with h/m-shc siRNA, p66-shc siRNA (from nt 236-256 in the CH2 domain, 5′-GAA UGA GUC UCU GUC AUC GUC-3′, SEQ ID NO:3; and 5′-CGA UGA CAG AGA CUC AUU CCG-3′, SEQ ID NO:4) and control luc siRNA essentially as described in Example 1. Whole-cell extracts were prepared 48 h later and analyzed for ShcA and β-tubulin levels as in Example 1. When cells were transfected with p66-shc siRNA, only the p66ShcA isoform was reduced with kinetics similar to that obtained with h/m-shc siRNA; the other two isoforms were not affected. Similarly, β-tubulin levels were not affected. The level of p66ShcA in cells challenged a second time with the p66-shc siRNA 6 days after the initial transfection, when the level of p66ShcA was very low but about to increase, and 10 days after the initial transfection, when the level of p66ShcA had recovered substantially, remained low and decreased markedly again after transfection at days 6 and 10, respectively.

This example illustrates the specificity of the siRNA effect with the effect of p66-sch si RNA being restricted to the p66ShcA isoform. Expression of p52/p46 ShcA was not affected although p66 and p52/p46 mRNAs share sequence identity in most of the region 3′ of the siRNA site, indicating that the silencing signal does not propagate to regions of mRNA 3′ to the siRNA in mammalian cells.

In addition, the sequence of p66-shc siRNA is present in the primary transcripts of p52/p46 ShcA mRNA but is spliced out of the mature mRNA. Although not wishing to be bound by theory, the fact that only the p66 ShcA isoform was downregulated by p66-shc siRNA strongly suggests that the site of action of siRNA is confined to the cytoplasm and to the spliced mRNA. If siRNA acted in the nucleus and triggered the decay of transcripts containing the corresponding sequence, the p52ShcA and p46ShcA proteins should have been equally downregulated by the same p66-shc siRNA, which was not the case.

### Example 3

### Isoform-Specific Expression of ShcA

This Example illustrates a rapid, alternative approach for isoform-specific gene expression. The previous Examples show how the adaptor protein ShcA can be suppressed in an isoform-specific manner in a human cell line. The siRNA with a sequence shared by the two ShcA transcripts suppresses p66, p52 and p46 (see Example 1). However, an siRNA whose sequence is present only in p66 mRNA suppresses only the p66 isoform (see Example 2), suggesting that the siRNA signal does not propagate to the 3′ region of the target mRNA. In this Example, the expression of individual isoforms is achieved by first downregulating all isoforms by the common (h/m shc) siRNA (as in Example 1) and then transfecting with an expression vector for the desired isoform harboring silent mutations at the site corresponding to the h/m-shc siRNA. This allows functional analylsis of individual ShcA isoforms or indeed any other gene encoding multiple proteins.

The full-length mouse p46, p52 and p66ShcA cDNAs were isolated from NIH3T3 cells by reverse transcriptase-polymerase chain reaction using the sense primers 5′-CGG AAT TCA TGG GAC CTG GGG TTT CCT ACT-3′ (SEQ ID NO:5), 5′-CGG AAT TCA TGA ACA AGC TGA GTG GAG GCG-3′ (SEQ ID NO:6) and 5′-CGG AAT TCA TGG ATC TTC TAC CCC CCA AGC CGA AGT A-3′ (SEQ ID NO:7), respectively, and the common antisense primer 5′-CGG AAT TCA CAC TTT CCG ATC CAC GGG TTG C-3′ (SEQ ID NO:8). Full-length ShcA cDNAs were initially cloned into pBluescriptll KS+ and nucleotide sequences verified by the dideoxynucleotide chain termination procedure.

An HA-tagged expression vector pcDNA3HA was constructed by inserting the overlapping oligonucleotide pair 5′-CCC ACC ATG GCT TAC CCA TAC GAT GTT CCA GAT TAC GCT G-3′ (SEQ ID NO:9) and 5′-AAT TCA GCG AAT TCT GGA ACA TCG TAT GGG TAA GCC ATG GTG GGG TAC-3′ (SEQ ID NO:10) into the Kpnl-EcoRI site of pcDNA3 (Invitrogen). To construct expression vectors for HA-tagged ShcA, p46HA, p52HA and p66HA, the full-length cDNAs of p46, p52 and p66 were inserted into the EcoRI-EcoRV site of pcDNA3HA. ShcA mutants in which potential internal initiation methionine codons were converted to leucine codons, thus expressing only the p66ShcA or 52ShcA forms, were created using the QuickChange site-directed mutagenesis kit (Stratagene). The overlapping oligonucleotide pair 5′-CTC CTC CAG GAC CTG AAC AAG CTG AGT G-3′ (SEQ ID NO:11) and 5′-CAC TCA GCT TGT TCA GGT CCT GGA GGA G-3′ (SEQ ID NO:12) was used to mutate methionine 65 (start site for p52) to leucine in p66HA, resulting in p66HA-m1. Another overlapping oligonucleotide pair, 5′-CCA ACG ACA AAG TCC TGG GAC CCG GGG-3′ (SEQ ID NO:13) and 5′-CCC CGG GTC CCA GGA CTT TGT CGT TGG-3′ (SEQ ID NO:14), was used to mutate the initiation sites for p46 in both p66HA-m1 and p52HA, resulting in p66HA-ML and p52HA-ML. Silent mutations were introduced into these vectors at the sites corresponding to h/m-shc siRNA as above using the overlapping oligonucleotide pair 5′-GGG GTT TCC TAC TTG GTC CGC TAC ATG GGT TGT C-3′ (SEQ ID NO:15) and 5′-CAC AAC CCA TGT AGC GGA CCA AGT AGG AAA CCC C-3′ (SEQ ID NO:16) to give p46HA-sm, p52HA-ML-sm and p66HA-ML-sm. Note that proteins expressed from these vectors are identical to the parent proteins.

For isoform-specific expression, HeLa cells were first transfected with no siRNA (mock) or with h/m-shc siRNA to downregulate all three endogenous ShcA proteins (isoforms) essentially as described above. Two days later, the cells were transfected with an empty expression vector, pCDNA3, or an expression vector for the desired isoform of wild-type mouse ShcA or silent mutant ShcA using Lipofectamine 2000 (Life Technologies) according to the manufacturer's instructions. One day later, whole-cell extracts were prepared and analyzed by Western blotting for the ShcA expression level. Membranes were blotted with polyclonal anti-ShcA and anti-β tubulin antibodies.

As already demonstrated in Example 1, h/m-shc siRNA knocked down all three isoforms of endogenous ShcA almost completely. Transfection of these cells with mutant expression vectors for individual isoforms resulted in the expression of only the corresponding isoforms. Almost no protein was detected in cells transfected with wild-type expression vectors demonstrating that the siRNA acts on the exogenous ShcA expressed by the vectors. Cells which were not transfected with h/m-shc siRNA expressed elevated levels of ShcA isoforms, irrespective of the presence or absence of mutations in the expression vectors. In contrast, siRNA did not act on the mutated sequences allowing isoform-specific expression of any Shc A isoform. Cells trasfected with p66HA-ML-sm clearly showed specific expression of only p66, whereas p46HA-sm and p52 HA-ML-sm resulted in expression of only p46 and p52, respectively.

The present inventors have shown that siRNA can efficiently, specifically and rapidly downregulate the level of an endogenous protein in mammalian cells. The effect of siRNA was restricted to mRNAs containing a sequence essentially identical to the siRNA used. That the primary action of siRNA on mRNA, which is most likely an endonucleolytic attack, does not propagate to other regions of the target mRNA was inferred from the following observations: (1) the effect of p66-shc siRNA was restricted to p66ShcA (Example 2) and (2) h/m-ShcA siRNA targeted wild-type ShcA mRNAs but not mutant mRNAs (Example 3). The second observation was with ectopically expressed ShcA mRNAs.

In this experiment, sequences of wild-type ShcA mRNA and mutant ShcA mRNA for each isoform were identical except for two nucleotides at the siRNA recognition site located in the middle of the mRNA. These two nucleotide cahgnes were sufficient to avoid the siRNA effect on the expression of the desired isoform. If the silencing signal would spread either 5′ or 3′ of the siRNA, ShcA expression from both wild-type and mutant mRNAs would have been suppressed, as at least some of these sequences would be identical. That expression from wild-type mRNAs but not from mutant mRNAs was suppressed strongly argues for stringent specificity of siRNA-mediated mRNA decay and no propagation of RNAi in mammalian cells. This is in stark contrast to results of experiments in an insect cell-free system (Lipardi et al., 2001, Cell 107, 297-307) and C. elegans (Sijen et al., 2001, Cell 107, 465-476) that have suggested that long dsRNA molecules synthesized by RNA-dependent RNA polymerase are intermediates in RNAi that amplify and maintain the effect of siRNA, implying 5′spreading of the silencing signal from siRNA. If amplification is involved in silencing in mammalian cells, antisense RNA oligonucleotides alone should serve as a primer for RNA-dependent RNA polymerase and, thus, be sufficient for gene downregulation. We found that antisense RNA did not induce silencing.

An important implication of our results is that the combination of PTGS using siRNA and the isoform-specific expression of an homologous gene with silent mutations causes the cell to express only one isoform, while keeping the levels of other isoforms very low. Using this knockdown-in method, it should be possible to examine the effect of various mutations of individual isoforms on cellular activity. The isoform sequences in the expression vectors should be designed so that they are not recognized by the siRNA affecting expression of the cellular genes. Two mismatches in the mutated expression vectors were sufficient for them to be free of siRNA-mediated suppression. We used expression vectors for mouse ShcA in this work because there is a high degree of sequence similarity between mouse and human ShcA (90% mRNA sequences and 93% amino acid sequences for p66 isoform). Thus, instead of introducing mutations into expression vectors, it would have been possible to design a different siRNA with a sequence matching perfectly the endogenous human ShcA gene but not the transfected mouse gene, and achieve similar results. The advantage of our approach, however, is that we can obtain isoform-specific ShcA expression in both human and mouse cells using the same set of probes.

In summary, we have shown in mammalian cells that the site of action of siRNA-mediated mRNA degradation is confined to the cytoplasm and that the target mRNA is restricted to those mRNAs containing a sequence essentially identical to the siRNA used. These specific features of siRNA-mediated gene knock-down can be employed over a short time period in conjunction with specific expression vectors to establish conditions for expression of the ShcA gene in an isoform-specific manner. This knockdown-in method should be applicable and useful for the study of any gene expressed as multiple isoforms.

The disclosures of each publication referred to herein, as well as priority application GB 0201477.7 filed Jan. 23, 2002, are hereby incorporated by reference as if each were referred to individually.

1. A method of expressing a desired isoform of a gene product in a cell absent undesired isoforms of a gene product, said method comprising:
(a) exposing a mammalian cell to at least one nucleic acid, said nucleic acid being at least a partially double-stranded ribonucleic acid and the double-stranded portion having at least 95% sequence identity to a common nucleic acid sequence shared by two or more isoforms of said gene product; and
(b) introducing an expression vector encoding a desired isoform of said gene product into said mammalian cell, said desired isoform having a sequence comprising one or more mismatches relative to said double-stranded portion of said nucleic acid, operably linked to a promoter capable of driving expression of said desired isoform in said cell.

2. The method of claim 1, wherein said common nucleic acid sequence is at least 19 consecutive nucleotides in length.

3. The method of claim 1, wherein said common nucleic acid sequence is common to all endogenous isoforms of said gene product in said cell.

4. The method of claim 1, wherein the double-stranded portion of said nucleic acid is 100% identical to said common nucleic acid sequence.

5. The method of claim 1, wherein said nucleic acid is 19 to 25 nucleotides long.

6. The method of claim 1, wherein said at least partially double-stranded ribonucleic acid comprises a double-stranded portion of at least 19 nucleotides and at least one two-nucleotide single-stranded 3′ overhang.

7. The method of claim 1, wherein said desired isoform comprises a sequence comprising two or more mismatches relative to said double-stranded portion of said nucleic acid.

8. The method of claim 1, wherein said expression vector encodes said desired isoform using at least one codon that differs from the endogenous sequence coding said desired isoform.

9. The method of claim 8, wherein said expression vector encodes said desired isoform using two codons that differ from the corresponding endogenous sequence coding said desired isoform.

10. The method of claim 8, wherein said desired isoform has an identical protein sequence to the corresponding endogenous isoform.

11. The method of claim 1, wherein said desired isoform replaces a mutant isoform in the cell.

12. The method of claim 11, wherein said mutant isoform is oncogenic, apoptotic, tumor suppressive, inflammation inducive or suppressive, or angiogenic.

13. The method of claim 1, further comprising determining the function of said desired isoform.

14. The method of claim 1, wherein said cell is a cancer cell.

15. The method of claim 14, wherein said cell is selected from the group consisting of HeLa (cervical cancer), PC3 (prostate cancer), MDA-MB-231 (breast cancer) and MCF-7.

16. The method of claim 1, wherein said desired isoform is transcribed under the control of an endogenous promoter.

17. The method of claim 1, wherein said expression vector comprises a constitutive promoter operably linked to said desired isoform.

18. The method of claim 1, wherein said expression vector comprises an inducible promoter operably linked to said desired isoform.

19. The method of claim 1, wherein said expression vector comprises a tissue-specific promoter operably linked to said desired isoform.

20. A kit comprising reagents expressing a desired isoform of a gene product in a cell absent undesired isoforms of a gene product, wherein said kit comprises a nucleic acid being at least a partially double-stranded ribonucleic acid and the double-stranded portion having at least 95% sequence identity to a common nucleic acid sequence shared by two or more isoforms of said gene product; and an expression vector encoding a desired isoform of said gene product, said desired isoform having a sequence comprising one or more mismatches relative to said double-stranded portion of said nucleic acid, operably linked to a promoter capable of driving expression of said desired isoform in said cell.

21. A mammalian cell exhibiting isoform-specific expression achieved by any of the methods of claim 1.

22. A method for treating a disease comprising administering to a subject in need of treatment an effective amount of a nucleic acid being at least a partially double-stranded ribonucleic acid and the double-stranded portion having at least 95% sequence identity to a common nucleic acid sequence shared by two or more isoforms of said gene product; and an expression vector encoding a desired isoform of said gene product, said desired isoform having a sequence comprising one or more mismatches relative to said double-stranded portion of said nucleic acid, operably linked to a promoter capable of driving expression of said desired isoform in said cell.

23. A method of assigning function to a desired isoform, said method comprising:
a) exposing a mammalian cell to at least one nucleic acid, said nucleic acid being at least a partially double-stranded ribonucleic acid and the double-stranded portion having at least 95% sequence identity to a common nucleic acid sequence shared by two or more isoforms of said gene product;
b) exposing said mammalian cell to an expression vector encoding a desired isoform of said gene product, said desired isoform having a sequence comprising one or more mismatches relative to said double-stranded portion of said nucleic acid, operably linked to a promoter capable of driving expression of said desired isoform in said cell;
c) identifying a phenotype of said mammalian cell compared to when said desired isoform is absent, and
d) assigning said phenotype or function to said desired isoform.


SEQUENCE LISTING (16 sequences)

SEQ ID NO: 1 - RNA, 21 nt, Artificial Sequence
  oligoribonucleotide pair used as siRNA; h/m-shc siRNA from nt 677-697 (in the PTB domain)
cuacuugguucgguacauggg

SEQ ID NO: 2 - RNA, 22 nt, Artificial Sequence
  oligoribonucleotide pair used as siRNA; h/m-shc siRNA from nt 677-697 (in the PTB domain)
cauguaccgaacccaaguagga

SEQ ID NO: 3 - RNA, 21 nt, Artificial Sequence
  oligonucleotide pair used for siRNA; h/m-shc siRNA, p66-shc siRNA (from nt 236-256 in the CH2 domain)
gaaugagucucugucaucguc

SEQ ID NO: 4 - RNA, 21 nt, Artificial Sequence
  oligonucleotide pair used in siRNA; h/m-shc siRNA, p66-shc siRNA (from nt 236-256 in the CH2 domain)
cgaugacagagacucauuccg

SEQ ID NO: 5 - DNA, 30 bp, Mus musculus
  primer_bind (1)...(30): sense primer for mouse p46 ShcA cDNA
cggaattcatgggacctggggtttcctact

SEQ ID NO: 6 - DNA, 30 bp, Mus musculus
  primer_bind (1)...(30): sense primer for mouse p52 ShcA cDNA
cggaattcatgaacaagctgagtggaggcg

SEQ ID NO: 7 - DNA, 36 bp, Mus musculus
  primer_bind (1)...(36): sense primer for mouse p66 ShcA cDNA
cggaattcatggatcttctaccccccaagccgaagt

SEQ ID NO: 8 - DNA, 31 bp, Mus musculus
  primer_bind (1)...(31): common antisense primer for ShcA cDNAs
cggaattcacactttccgatccacgggttgc

SEQ ID NO: 9 - DNA, 40 bp, Artificial Sequence
  overlapping oligonucleotide pair for insertion into the KpnI-EcoRI site of pcDNA3
cccaccatggcttacccatacgatgttccagattacgctg

SEQ ID NO: 10 - DNA, 48 bp, Artificial Sequence
  overlapping oligonucleotide pair for insertion into the KpnI-EcoRI site of pcDNA3
aattcagcgaattctggaacatcgtatgggtaagccatggtggggtac

SEQ ID NO: 11 - DNA, 27 bp, Artificial Sequence
  overlapping oligonucleotide pair used to mutate methionine 65 (start site for p52) to leucine in p66HA, resulting in p66HA-m1
ctcctccaggacctgaacaagctgagt

SEQ ID NO: 12 - DNA, 28 bp, Artificial Sequence
  overlapping oligonucleotide pair used to mutate methionine 65 (start site for p52) to leucine in p66HA, resulting in p66HA-m1
cactcagcttgttcaggtcctggaggag

SEQ ID NO: 13 - DNA, 27 bp, Artificial Sequence
  overlapping oligonucleotide pair used to mutate the initiation sites for p46 in both p66HA-m1 and p52HA, resulting in p66HA-ML and p52HA-ML
ccaacgacaaagtcctgggacccgggg

SEQ ID NO: 14 - DNA, 27 bp, Artificial Sequence
  overlapping oligonucleotide pair used to mutate the initiation sites for p46 in both p66HA-m1 and p52HA, resulting in p66HA-ML and p52HA-ML
ccccgggtcccaggactttgtcgttgg

SEQ ID NO: 15 - DNA, 34 bp, Artificial Sequence
  overlapping oligonucleotide pair used to give p46HA-sm, p52HA-ML-sm and p66HA-ML-sm
ggggtttcctacttggtccgctacatgggttgtc

SEQ ID NO: 16 - DNA, 34 bp, Artificial Sequence
  overlapping oligonucleotide pair used to give p46HA-sm, p52HA-ML-sm and p66HA-ML-sm
cacaacccatgtagcggaccaagtaggaaacccc